(19) United States
(12) Patent Application Publication
(10) Pub. No.: US 2026/0192237 A1
(43) Pub. Date: Jul. 9, 2026

(54) INTEGRATED ZEOLITE MEMBRANE-REACTOR AND ADSORPTION PROCESS FOR CO2 CAPTURE AND CONVERSION TO METHANOL

(71) Applicants: Jerry Lin, Scottsdale, AZ (US); Shuguang Deng, Mesa, AZ (US)

(72) Inventors: Jerry Lin, Scottsdale, AZ (US); Shuguang Deng, Mesa, AZ (US)

(21) Appl. No.: 19/437,911

(22) Filed: Dec. 31, 2025

Related U.S. Application Data
(60) Provisional application No. 63/741,621, filed on Jan. 3, 2025.

(51) Int. Cl. B01D 53/04; B01D 53/00; B01J 19/24; C07C 29/151; C07C 29/152; C07C 29/76

(52) U.S. Cl. CPC B01D 53/0462; B01D 53/002; B01J 19/2415; B01J 19/2475; C07C 29/1518; C07C 29/152; C07C 29/76; B01D 2253/104; B01D 2253/108; B01D 2257/504; B01D 2257/80; B01D 2258/0283; B01D 2259/402

(57) ABSTRACT

A carbon dioxide capture and conversion system includes a temperature swing adsorption unit configured to capture carbon dioxide and water and a zeolite membrane reactor configured to convert carbon dioxide into a product including methanol. A zeolite membrane reactor includes a housing, a plurality of hollow fiber zeolite membrane tubules arranged in the housing with each hollow fiber zeolite membrane defining an annulus, and a carbon dioxide hydrogenation catalyst contained in the annulus. Capturing and converting carbon dioxide includes providing an input stream including carbon dioxide to a temperature swing adsorption unit, separating the input stream into a carbon dioxide-rich stream and a carbon dioxide-lean stream, providing the carbon dioxide-rich stream and hydrogen gas to a zeolite membrane reactor, and hydrogenating the carbon dioxide-rich stream to yield a retentate stream including methanol and a permeate stream including water and nitrogen gas.

### CROSS-REFERENCE TO RELATED APPLICATION

This application claims the benefit of U.S. Patent Application No. 63/741,621 filed on Jan. 3, 2025, which is incorporated by reference herein in its entirety.

### STATEMENT OF GOVERNMENT SUPPORT

This invention was made with government support under CBET-2200204 awarded by the National Science Foundation. The government has certain rights in the invention.

### TECHNICAL FIELD

This invention relates to a method of carbon dioxide (CO₂) capture and conversion into methanol.

### BACKGROUND

Carbon capture technologies can reduce atmospheric carbon dioxide (CO₂). Captured CO₂ can be repurposed for carbon utilization, converting waste into resources and supporting a transition to a low-carbon, circular economy. Methanol is a feedstock for synthesizing various chemical products and an energy storage medium due at least in part to its high energy density, storability, and transportability. It can store electric energy from renewable sources or fossil power plants through CO₂ hydrogenation.

### SUMMARY

This disclosure describes an integrated zeolite membrane-reactor and adsorption process that captures carbon dioxide (CO₂) from coal-burning flue gas and converts it into methanol. The system includes a zeolite-based temperature swing adsorption unit for capturing CO₂ and water (H₂O) and a zeolite membrane reactor for converting the captured CO₂, with green hydrogen, into methanol. Waste heat and recycled gas from the zeolite membrane reactor are used to regenerate the zeolite adsorbents in the temperature swing adsorption unit.

In a first general aspect, a carbon dioxide capture and conversion system includes a temperature swing adsorption unit configured to capture carbon dioxide and water and a zeolite membrane reactor configured to convert the carbon dioxide into a product including methanol.

Implementations of the first general aspect may include one or more of the following features.

In some cases, the temperature swing adsorption unit includes zeolite adsorbents. The first general aspect can further include a recycle loop configured to provide waste heat and recycled gas from the zeolite membrane reactor to the temperature swing adsorption unit to regenerate the zeolite adsorbents. In some implementations, the temperature swing adsorption unit includes a double-layer adsorbent column. The double-layer adsorbent column can include activated alumina and zeolite. In some cases, the temperature swing adsorption unit includes two columns. The first general aspect can further include an electrolysis unit configured to provide hydrogen gas to the zeolite membrane reactor. In some implementations, the first general aspect further includes a condenser configured to receive a gas mixture from the zeolite membrane reactor and to recover methanol from the gas mixture. The first general aspect can further include a recycle loop configured to provide a gas phase output stream from the condenser to the temperature swing adsorption unit as a purge gas. In some cases, the first general aspect further includes a condenser fluidly coupled to the zeolite membrane reactor and configured to recover water from a stream including water and nitrogen.

In a second general aspect, capturing and converting carbon dioxide includes providing an input stream including carbon dioxide to a temperature swing adsorption unit, separating the input stream including carbon dioxide into a carbon dioxide-rich stream and a carbon dioxide-lean stream, providing the carbon dioxide-rich stream and hydrogen gas to a zeolite membrane reactor, and hydrogenating the carbon dioxide in the carbon dioxide-rich stream to yield a retentate stream including methanol and a permeate stream including water and nitrogen gas.

Implementations of the second general aspect may include one or more of the following features.

In some implementations, the input stream includes flue gas. The carbon dioxide-rich stream can further include carbon monoxide and additional hydrogen gas. In some cases, the second general aspect further includes separating the retentate stream into a product stream including methanol and a recycle stream including carbon dioxide, carbon monoxide, and hydrogen gas. The second general aspect can further include providing the recycle stream to the temperature swing adsorption unit as a purge gas. In some implementations, the second general aspect further includes separating the permeate stream into a stream including water and a stream including nitrogen gas. In some cases, the second general aspect can further include providing the carbon dioxide-lean stream to the zeolite membrane reactor as a sweep gas. The second general aspect can further include producing the hydrogen by electrolysis. In some implementations, producing the hydrogen includes powering the electrolysis with a renewable energy source.

In a third general aspect, a zeolite membrane reactor includes a housing, a plurality of hollow fiber zeolite membrane tubules arranged in the housing, wherein each hollow fiber zeolite membrane tubule of the plurality of hollow fiber zeolite membrane tubules defines an annulus, and a carbon dioxide hydrogenation catalyst contained in the annulus.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 is a block flow diagram of an integrated sorption and membrane reactor system for carbon dioxide (CO₂) capture and conversion.

FIG. 2 shows equilibrium CO₂ conversion at different temperatures and total pressures for CO₂ hydrogenation to methanol.

FIG. 3 is a schematic diagram showing a single tube zeolite membrane reactor for CO₂ hydrogenation where catalyst can be either packed in the bore (as shown) or the annulus of the membrane tube.

FIG. 4 is a block flow diagram of an integrated temperature swing adsorption CO₂ capture unit with zeolite membrane reactor for CO₂ hydrogenation.

FIG. 5 shows modeling results on the performance of H₂O-selective zeolite membrane reactors at different temperatures (fixed pressure at 35 bar).

FIG. 6 is a schematic of an example membrane reactor module with catalyst in a bench-scale system.

FIG. 7 is a process flow diagram for an example reactive CO₂ capture and conversion process.

FIG. 8 is a flow chart showing operations in a process to capture and convert carbon dioxide.

### DETAILED DESCRIPTION

This disclosure describes the integration of a zeolite-based membrane reactor and a temperature swing adsorption system for carbon dioxide (CO₂) capture and conversion into methanol. The zeolite membrane reactor enhances CO₂ conversion and enables product separation, while the integration of temperature swing adsorption leads to CO₂ capture and regeneration. Captured CO₂ can be converted into methanol, providing a pathway for carbon utilization rather than sequestration. The temperature swing adsorption and zeolite membrane reactor units are integrated to optimize heat and mass transfer between the two systems.

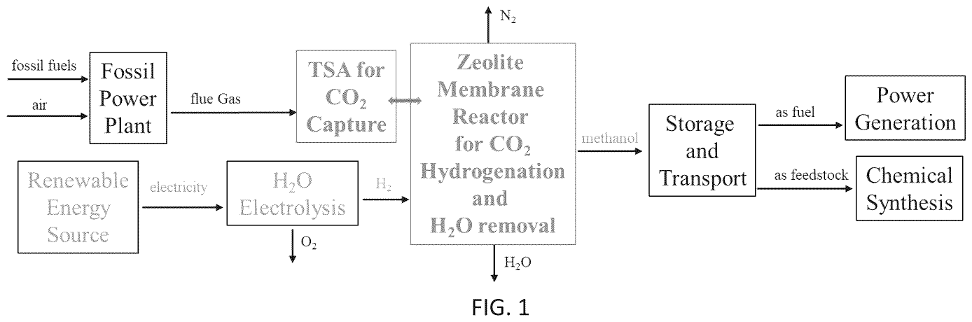

FIG. 1 is a block flow diagram of an example CO₂ capture and conversion system that includes a zeolite-based temperature swing adsorption unit and a zeolite membrane reactor. The zeolite-based temperature swing adsorption unit captures CO₂ and H₂O and the zeolite membrane reactor converts the captured CO₂, with green hydrogen, into methanol. Waste heat and recycled gas from the zeolite membrane reactor is used to regenerate the zeolite adsorbents in the temperature swing adsorption unit, and the clean nitrogen gas (N₂) produced in the temperature swing adsorption unit is used as sweep gas in the zeolite membrane reactor unit, improving system energy efficiency. Green hydrogen can be supplied by an electrolysis unit powered by renewable energy sources. Outputs of the system include methanol (e.g., as the primary product) along with CO₂-lean water and nitrogen (e.g., water and nitrogen that is free or substantially free of CO₂).

CO₂ is converted to methanol through CO₂ hydrogenation which involves the following reactions:

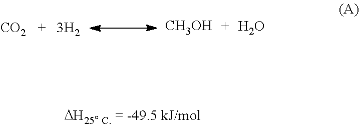

The CO₂ hydrogenation is accompanied by the following two series/parallel side reactions:

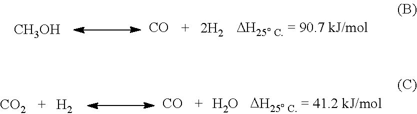

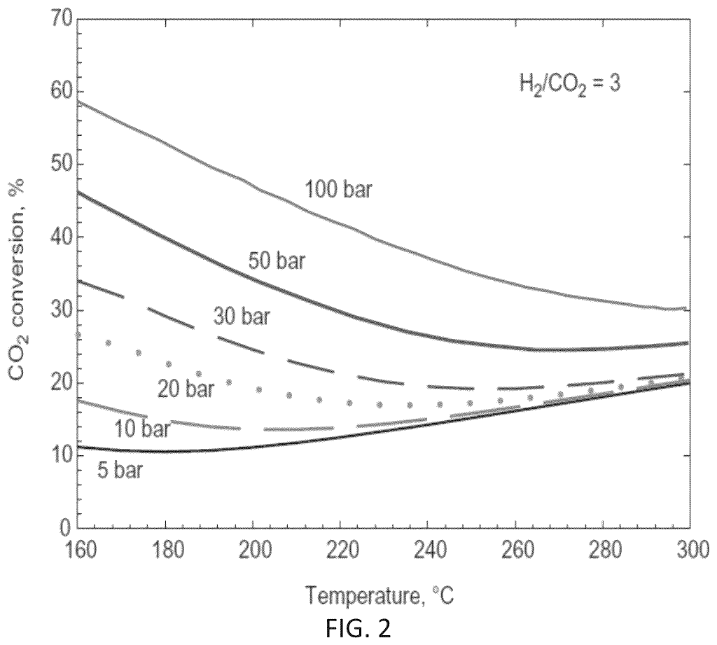

Stoichiometrically, in Reaction (A), a third of the H₂ is converted to water. The side Reaction (C) also consumes hydrogen to form water. CO, the main side product, is formed through Reaction (B) and Reaction (C). Thermodynamically, methanol formation (Reaction (A)) is favored at high pressure and low temperature, but the actual conversion also depends on the kinetic of the reactions, as shown in FIG. 2. The equilibrium CO₂ conversion at 10 bar is lower than 20% and can reach about 60% at 100 bar and 160° C. Above 30 bar, lowering the reaction temperature increases equilibrium conversion but reduces the reaction kinetics.

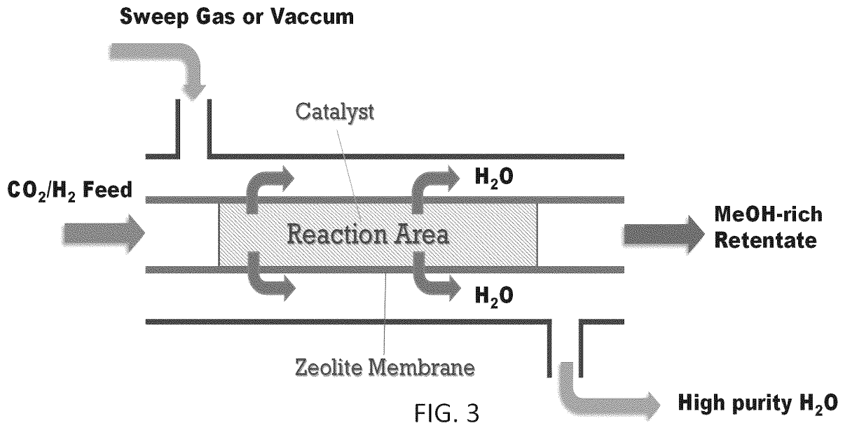

Membrane reactors can be used for chemical reactions with low conversion limited by thermodynamic equilibrium. In a membrane reactor, a water-selective inorganic membrane tube is packed with a CO₂ hydrogenation catalyst, as shown in FIG. 3. During the reaction, one of the products (e.g., H₂O) permeates through the membrane, enhancing conversion.

In some examples, the composition of flue gas from a coal-fired power plant, after flue gas desulfurization, includes 13% CO₂, 73% N₂, 4% O₂, and 10% H₂O at 1 atm and a temperature between 40° C. and 60° C. Unless specified otherwise, the gas percentages are expressed as molar percent (mol %). CO₂-selective membranes can offer the potential to be combined with CO₂ hydrogenation reactors, enabling CO₂ capture from flue gas and in-situ reaction with hydrogen in the reactor to form methanol.

A temperature swing adsorption unit integrated with a zeolite membrane reactor uses adsorption-based technology to capture CO₂ from flue gas. In some examples, the temperature swing adsorption unit utilizes commercial zeolite 13X (APG from UOP/Honeywell) as the adsorbent. Zeolite 13X exhibits a CO₂ adsorption capacity of 3.5 mmol/g at a CO₂ partial pressure of 0.13 atm and 45° C., an ideal adsorbed solution theory (IAST) CO₂/N₂ separation selectivity greater than 100 at 0.13 atm CO₂ and 25° C., and a CO₂ heat of adsorption of 37 kJ/mol. To protect the CO₂ adsorption capacity of the zeolite in flue gas capture, a dual-layer bed configuration can be employed. In some cases, the bottom layer is activated alumina (BASF F-200) and the top layer is zeolite 13X (APG from UOP/Honeywell).

Example operating conditions for the temperature swing adsorption CO₂ capture process are provided in Table 1. CO₂ from the flue gas can be captured at near ambient temperature and desorbed at 250° C. The temperature swing adsorption unit can include two adsorbers—one for adsorption operated at ambient temperature, and the other for desorption operated at 250° C. (e.g, the same as the zeolite membrane reactor). Both adsorption and desorption can be operated near ambient pressure. These conditions can allow the integration of the CO₂ capture unit with the zeolite membrane reactor for CO₂ conversion, leveraging synergistic heat and mass transfer between the systems.

TABLE 1
Temperature swing adsorption process conditions for CO₂ capture

| | | | Depressurization | Desorption with hot | | Pressurization |
|---|---|---|---|---|---|---|
| | Adsorption | | and heating | gas purging | | and cooling |
| Temperature | 25° | C. | 25° C. → 250° C. | 250° | C. | 250° C. → 25° C. |
| Pressure | 1.5 | bar | 1.5 bar → 1 bar | 1 | bar | 1 bar → 1.5 bar |
| CO₂ concentration | 13% | | | 47% | | |
| Duration | 300 | seconds | 150 seconds | 900 | seconds | 450 seconds |

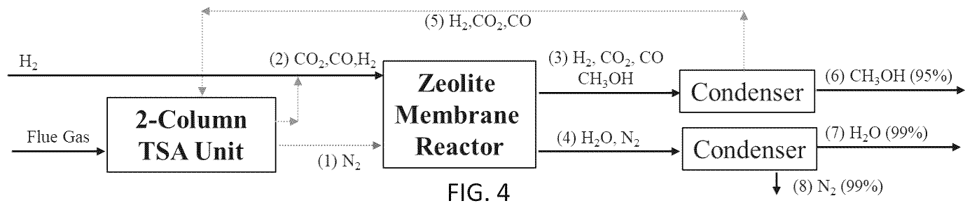

FIG. 4 is a block flow diagram of an example integrated zeolite membrane reactor and adsorption process. Clean, near-room-temperature flue gas from a fossil fuel power plant is directed to a two-column temperature swing adsorption unit for CO₂ capture, producing a cold, CO₂-lean gas stream (primarily N₂) at 1 atm (Stream 1). The temperature swing adsorption unit captures CO₂, which can subsequently be released from the adsorbent bed as a CO₂-rich stream mixed with CO and H₂ (Stream 2). This desorption stream is generated by purging with a hot gas stream (Stream 5). The hot gas stream (Stream 5) includes unreacted CO₂, H₂, and byproduct H₂ and CO separated from the retentate stream of the zeolite membrane reactor (Stream 3) via a condenser.

The warm CO₂-rich stream (Stream 2) and additional H₂ is fed into the zeolite membrane reactor at an H₂/CO₂ molar ratio of 3, operating at approximately 35 bar and 250° C. Water vapor permeates to the permeate side of the zeolite membrane reactor, which is maintained at 1 atm, while N₂ from the temperature swing adsorption unit (Stream 1) acts as the sweep gas. The nearly water-free retentate stream (Stream 3) from the zeolite membrane reactor passes through a condenser to produce methanol with a purity exceeding 95% (Stream 6). The gas stream from the condenser (Stream 5) is heated and utilized to purge the adsorbed CO₂ at approximately 250° C. in the temperature swing adsorption unit. The permeate from the zeolite membrane reactor, containing N₂ (with a small amount of O₂) and H₂O, is separated using a condenser, resulting in two pure streams: H₂O (Stream 7) and N₂ (Stream 8). The integrated temperature swing adsorption and zeolite membrane reactor process includes two feed streams—flue gas from the power plant and green H₂ from renewable sources—and three product streams: methanol, water, and nitrogen. This configuration represents a process for CO₂ capture and conversion into methanol.

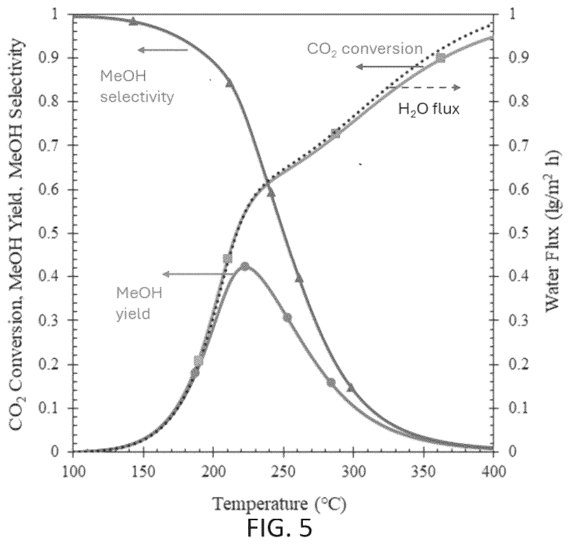

A mathematical model is developed to analyze CO₂ hydrogenation in the Linde Type A zeolite membrane reactor, incorporating CO₂ hydrogenation reaction kinetics and gas permeation equations validated by various measurements. The zeolite membrane reactor's performance at various operating temperatures is shown in FIG. 5. The results indicated that 220° C. provided a methanol yield of 43% and selectivity of 78%, as shown in Table 2. The simulation results suggest that, due at least in part to the high water vapor selectivity of the zeolite membrane, the permeate contains more than 99% water while the retentate contains about 0.5% water.

TABLE 2
Membrane reactor assessments for CO₂ hydrogenation to methanol

| | Membrane reactor |
|---|---|
| | assessments |
| Operation temperature (° C.) | 200-250 |
| Operation pressure (bar) | 30-40 |
| Catalyst amount (g) | 3-15 |
| CO₂ feed flow rate (ml (STP)/min)@ | 50-250 |
| H₂ feed flow rate (ml (STP)/min) | 150-750 |
| Gas hourly space velocity (GHSV) (1/hr) | 4,000-15,000 |
| Membrane area (cm²) | 20-100 |
| CO₂ Conversion | 55%# |
| MeOH yield | 43%# |
| MeOH Selectivity | 78%# |

#Modeling results are obtained at 220° C.

Water-selective Linde Type A zeolite membranes can be used in industrial processes for organic solvent dehydration. These membranes operate in pervaporation mode, where water selectively permeates through the membranes with a high water-to-organic separation factor.

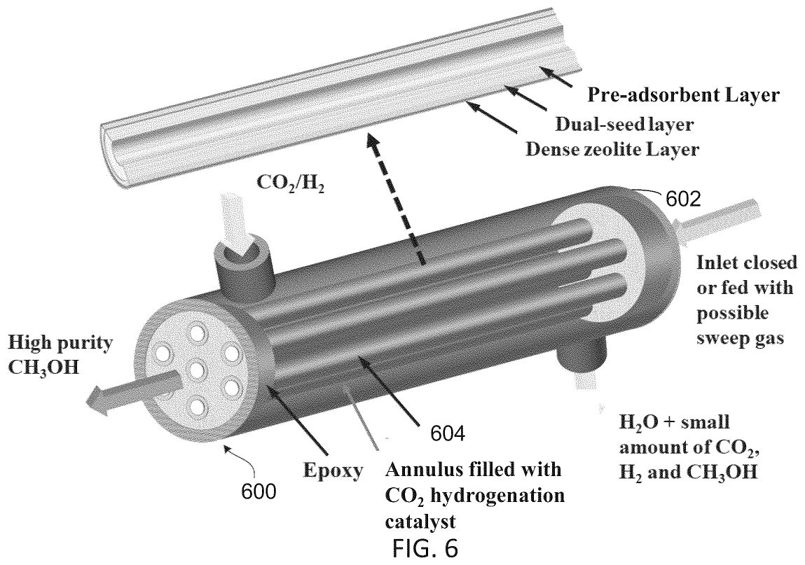

The zeolite membrane reactor module can be designed to resemble a multi-tube heat exchanger. FIG. 6 is a schematic diagram of an example zeolite membrane reactor 600. The zeolite membrane reactor 600 includes a housing 602 and a plurality of hollow fiber zeolite membrane tubules 604 arranged in the housing 602. Each hollow fiber zeolite membrane tubule of the plurality of hollow fiber zeolite membrane tubules 604 defines an annulus.

In some cases, the plurality of zeolite membrane tubules 604 are housed in a metal casing with high-temperature epoxy. The annulus of the plurality of zeolite tubules 604 is packed with a CO₂ hydrogenation catalyst. High-pressure CO₂/H₂ is typically fed into the annulus, where methanol produced in the reaction permeates into the bore of the membrane tubules and can be collected at the permeate outlet (with or without a sweep gas). Some of the plurality of zeolite tubules 604 can be replaced with metal ones to facilitate heat exchange and remove reaction heat. The number and dimensions of the plurality of zeolite membrane tubules 604 can be determined using modeling analysis. In some examples, hollow fiber zeolite membranes approximately 15 cm in length and a module with seven hollow fibers provide sufficient membrane surface area.

The membrane reactor system allows feeding of a high-pressure, high-temperature CO₂/H₂ mixture at controlled flow rates into the membrane reactor module. The gas composition and flow rates of the retentate and permeate are evaluated. With N₂ as the sweep gas at 1 am, the CO₂ conversion, selectivity, and methanol yield (and space-time yield) under various temperatures, pressures, and flow rates, are assessed for the catalyst-packed zeolite membrane reactor. These results can identify operating conditions for efficient methanol production.

A mathematical model captures characteristics of the zeolite membrane reactor for CO₂ hydrogenation to methanol, as shown in FIG. 5. The zeolite membrane reactor is operated with a Cu/ZnO/Al₂O₃ catalyst packed in the annulus of the reactor module. Reference kinetic equations for CO₂ hydrogenation to methanol (Reaction (A)) and the side reactions on the heterogenous catalyst (Reactions (B) and (C)) are used. For non-isothermal operation, a heat balance can yield two differential equations governing heat transfer in the annulus and bore of the membrane module. These differential equations, along with the kinetic, permeation flux, and heat-transfer equations, can be solved numerically using matrix laboratory (MATLAB). The model solutions provide partial pressure profiles along the reactor's axial direction (z), allowing the performance of the membrane reactor to be quantified in terms of CO₂ conversion, methanol selectivity, yield, space-time yield, and methanol purity in the permeate.

The effects of various operating parameters (temperature, pressure, space velocity, feed composition, membrane permeance, and selectivity) on the reactor's performance, including CO₂ conversion, methanol selectivity, and yield are numerically assessed. These parametric measurements can provide insight into which operating parameters of the membrane lead to maximum methanol yield and purity.

Cycle times for adsorption and desorption phases to maximize CO₂ capture efficiency and minimize energy consumption are assessed by balancing the adsorption capacity of zeolite adsorbents (e.g., zeolite 13X) with the regeneration efficiency during desorption. Advanced heat integration strategies, such as using waste heat from the zeolite membrane reactor to supply thermal energy for desorption is evaluated. This can reduce the external energy demand of the temperature swing adsorption process and improve its overall efficiency. Alternative regeneration methods, such as vacuum-assisted desorption or hybrid heating techniques, can enhance the regeneration of the zeolite adsorbents while reducing energy consumption during the desorption phase.

A CO₂ concentration of 40% to 50% is typically sufficient for a zeolite membrane reactor. Flue gas pre-treatment steps (e.g., humidity control or removal of contaminants such as sulfur oxides and nitrogen oxides) to prevent zeolite adsorbent degradation over time and improve CO₂ adsorption performance are assessed. The use of a dual-layer adsorption bed, including zeolite 13X combined with a pre-adsorbent layer (e.g., activated alumina) to handle moisture in the flue gas, is evaluated for its potential to improve overall CO₂ capture performance and protect the zeolite from deactivation. Process simulation and scaled-up assessments are conducted to determine the energy and materials for large-scale temperature swing adsorption systems. This provides insight into the industrial feasibility of using zeolite 13X for large-scale CO₂ capture from power plant flue gas.

TABLE 3
Two example temperature swing adsorption units for CO₂ capture

| | Temperature | Temperature |
|---|---|---|
| | swing adsorption | swing adsorption |
| | unit #1 | unit #2 |
| Adsorbent amount (g) | 47 | 300 |
| Adsorption temperature (° C.) | 30 | 45 |
| Adsorption pressure (bar) | 1.3 | 1.3 |
| Desorption Temperature (° C.) | 170 | 250 |
| Desorption pressure (bar) | 0.01-1 | 0.01, 1.3 |
| Superficial velocity at adsorption | 0.2 | 0.3 |
| (m/s) | | |
| Superficial velocity at desorption | 0.1 | 0.2 |
| (m/s) | | |
| CO₂ production rate (kg CO₂/hr) | 4 | >25 |
| CO₂ concentration (%) | 66-94% | 40-50% |
| CO₂ recovery (%) | 78-97% | >95% |

Table 3 summarizes the operating conditions and performance metrics of two example temperature swing adsorption units, temperature swing adsorption unit #1 and temperature swing adsorption unit #2. The scale of the temperature swing adsorption unit #2 is larger than that of temperature swing adsorption unit #1. Table 3 shows that CO₂ capture occurs for both temperature swing adsorption unit #1 (78-97%) and temperature swing adsorption unit #2 (>95%).

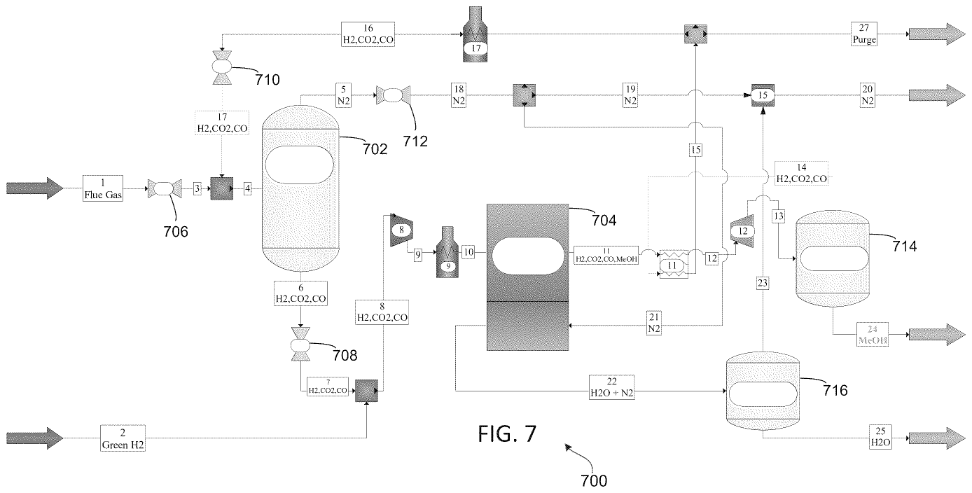

FIG. 7 is a process flow diagram of an example integrated CO₂ capture and conversion system 700. The CO₂ capture and conversion system 700 includes a temperature swing adsorption unit 702 and a zeolite membrane reactor 704. The CO₂ capture and conversion system 700 takes in flue gas generated from a coal-fired power plant and green hydrogen sourced from renewable energy. The outputs of this system include high-purity methanol, which serves as a fuel and chemical feedstock, along with high-purity water and nitrogen. This integrated approach can mitigate CO₂ emissions and promote the use of renewable energy to produce valuable resources.

Flue gas is fed into the temperature swing adsorption unit 702 for the capture of CO₂ and H₂O. The temperature swing adsorption unit 702 utilizes a double-layer adsorbent column, featuring a pre-adsorbent and a zeolite adsorbent layer. In some cases, the pre-adsorbent layer is activated alumina and the zeolite adsorbent layer is zeolite 13X, and operates at ambient temperature and about 1 bar for adsorption and 250° C. and 1 bar for desorption.

During the adsorption phase, with valves 706 and 712 open and valves 708 and 710 closed, the system 700 produces a CO₂-lean dry air stream (94.8% N₂+5.2% O₂), as shown in FIG. 7. A portion of this CO₂-lean air (Stream 5) is used as the sweep gas in the zeolite membrane reactor. The temperature swing adsorption unit 702 includes double-layer adsorbent columns. In some cases, the double-layer adsorbent columns include alumina and zeolite. In some cases, the temperature swing adsorption unit 702 includes two columns. Using a two-column configuration can lead to a continuous CO₂ and CO₂-lean dry air flow to the zeolite membrane reactor, allowing for uninterrupted operation of the integrated system.

In the regeneration phase, when valves 708 and 710 are open and valves 706 and 712 are closed, the captured CO₂ and H₂O is desorbed by a purge gas stream (Stream 15). The CO₂ capture and conversion system 700 further includes a recycle loop configured to provide waste heat and recycled gas from the zeolite membrane reactor 704 to the temperature swing adsorption unit 702 to regenerate the zeolite adsorbents.

In some examples, the purged gas stream, heated to 250° C. at 1 bar, is recycled from the zeolite membrane reactor 704 to release the adsorbed components. The resulting regeneration gas (Stream 6), containing CO₂, H₂O, H₂, and CO, is fed into the zeolite membrane reactor 704 along with the green hydrogen (Stream 2) produced via water electrolysis using renewable energy sources like wind and solar power. The feed gas (Stream 8) is compressed to 30 bar and heated to approximately 250° C. before being introduced into the zeolite membrane reactor 704. The CO₂ capture and conversion system 700 can further include an electrolysis unit configured to provide hydrogen gas to the zeolite membrane reactor 704.

The water produced in the reaction can selectively permeate across the membrane into the shell side, as shown in FIG. 3. As a result, the product on the tube side is a water-lean (e.g., water free or substantially water free) dry gas mixture containing CH₃OH, H₂, CO₂, and CO (Stream 11), as shown in FIG. 7. This mixture undergoes heat exchange with Stream 14, followed by an expansion to reduce the pressure from 30 bar to 1 bar. The mixture is condensed in a condenser 714 to recover methanol from the gas mixture. The gas phase exiting the condenser (Stream 14) is subsequently heated by both the gas product from the membrane reactor (Stream 11) and an external fire heater to approximately 250° C., before being introduced into the temperature swing adsorption unit as a purge gas during the regeneration phase to recover the captured CO₂. Meanwhile, the permeation product, which contains water and N₂ (Stream 22), is condensed in a condenser 716 to recover the high-purity water as a coproduct (Stream 25). For reaction operations below 300° C., various polymer sealing materials with flexible designs can be used in sealing the membrane module for mechanical stability and gas tightness. In some cases, epoxy is used.

For stability of the zeolite membrane under operational conditions, including high pressure, intermediate temperatures, and exposure to steam, the Si/Al ratio can be increased. Catalyst performance is monitored by regularly regenerating or replacing catalysts and conducting in situ characterization. Computational fluid dynamics modeling is used to monitor pressure drops, mass transfer limitations, and flow distribution for the integrated zeolite membrane reactor with the adsorption system.

Gas/vapor permeation and separation of the membranes are tested with a feed of equal molar CO₂, CO, H₂, and H₂O vapor at a temperature between 150° C. and 250° C. The flux equations for each gas for mixture permeation for the zeolite membranes is obtained. Values for permeation coefficients for the flux equations are obtained during measurements.

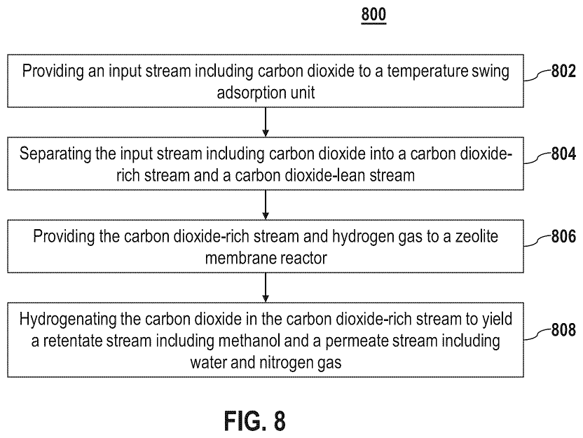

FIG. 8 is a flow chart showing operations in example process 800 of capturing and converting carbon dioxide. In 802, an input stream including carbon dioxide is provided to a temperature swing adsorption unit. The input stream can include flue gas. In 804, the input stream including carbon dioxide is separated into a carbon dioxide-rich stream and a carbon-dioxide lean stream. The carbon dioxide-rich stream can further include carbon monoxide and additional hydrogen gas. In 806, the carbon dioxide-rich stream and hydrogen gas is provided to a zeolite membrane reactor. In 808, the carbon dioxide in the carbon dioxide-rich stream is hydrogenated to yield a retentate stream including methanol and a permeate stream including water and nitrogen gas. The process 800 can further include separating the retentate stream into a product stream including methanol and a recycle stream including carbon dioxide, carbon monoxide, and hydrogen gas. In some implementations, the process 800 further includes providing the recycle stream to the temperature swing adsorption unit as a purge gas. In some cases, the process 800 further includes separating the permeate stream into a stream including water and a stream including nitrogen gas. The process 800 can further include providing the carbon dioxide-lean stream to the zeolite membrane reactor as a sweep gas. In some cases, the hydrogen is produced by electrolysis. The production of hydrogen can include powering the electrolysis with a renewable energy source.

Modeling analysis of a zeolite membrane reactor: A mathematical model is developed including differential mass and heat balance equations for the annulus (reaction chamber) and bore (permeation chamber) of the multi-tube zeolite membranes, gas permeation flux equations, and kinetic equations for CO₂ hydrogenation using a Cu/ZnO/Al₂O₃ catalyst. Reference kinetic equations, including reaction rate constants for both the main and side reactions are used. Due at least in part to the integration with the temperature swing adsorption process, the reactor feed is a mixture of CO₂, CO, and H₂, rather than a 1:3 CO₂/H₂ ratio. The model equations are non-dimensionalized, and MATLAB codes are generated to solve them numerically. The model's validity can be verified through a macroscopic mass balance of the reaction and permeate streams.

Modeling analysis is conducted to evaluate the impact of operational conditions on a bench-scale zeolite membrane reactor for CO₂ hydrogenation to methanol. The performance of the membrane reactor is characterized by metrics such as methanol yield and selectivity, space-time yield, methanol permeation flux, permeate methanol purity, and retentate composition (e.g., water vapor, with trace amounts of CO₂, H₂, and methanol). The model is used to assess the effects of membrane permeance and selectivity, reactor pressure, temperature, space velocity, and feed composition on these performance characteristics. Conditions are determined to maximize methanol yield, space-time yield, and methanol purity.

Modeling analysis of an integrate membrane reactor and adsorption system: A mathematical model that captures heat and mass transfer dynamics in the adsorption and desorption bed is established and solved numerically. This involves solving coupled differential equations for heat conduction, adsorption kinetics, and gas diffusion inside the porous structure of zeolites. The Langmuir or Freundlich isotherms can be used to describe adsorption equilibrium, while temperature-dependent kinetic models account for the desorption phase during the heating step. The modeling is focused on the step of desorption using retentate gas containing CO₂, CO and H₂. Parameters to consider include the thermal conductivity of the zeolite, the energy for desorption, and the cycle time. Simulation of the temperature swing adsorption process uses numerical methods and software such as MATLAB to assess system performance and predict gas capture efficiency under varying operational conditions.

The performance of an integrated zeolite membrane reactor and temperature swing adsorption process is assessed. The retentate stream after the removal of methanol is used as the sweep stream for the desorption column of the temperature swing adsorption unit, and the low-pressure CO₂-lean stream from the adsorption column of the temperature swing adsorption unit is used to sweep the permeate side of the zeolite membrane reactor. Model analysis is conducted to identify operation conditions for the zeolite membrane reactor and temperature swing adsorption that lead to high-purity methanol, water, and nitrogen streams production.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A carbon dioxide capture and conversion system comprising:
a temperature swing adsorption unit configured to capture carbon dioxide and water; and
a zeolite membrane reactor configured to convert the carbon dioxide into a product comprising methanol.

2. The carbon dioxide capture and conversion system of claim 1, wherein the temperature swing adsorption unit comprises zeolite adsorbents.

3. The carbon dioxide capture and conversion system of claim 2, further comprising a recycle loop configured to provide waste heat and recycled gas from the zeolite membrane reactor to the temperature swing adsorption unit to regenerate the zeolite adsorbents.

4. The carbon dioxide capture and conversion system of claim 1, wherein the temperature swing adsorption unit comprises a double-layer adsorbent column.

5. The carbon dioxide capture and conversion system of claim 4, wherein the double-layer adsorbent column comprises activated alumina and zeolite.

6. The carbon dioxide capture and conversion system of claim 1, wherein the temperature swing adsorption unit comprises two columns.

7. The carbon dioxide capture and conversion system of claim 1, further comprising an electrolysis unit configured to provide hydrogen gas to the zeolite membrane reactor.

8. The carbon dioxide capture and conversion system of claim 1, further comprising a condenser configured to receive a gas mixture from the zeolite membrane reactor and to recover methanol from the gas mixture.

9. The carbon dioxide capture and conversion system of claim 8, further comprising a recycle loop configured to provide a gas phase output stream from the condenser to the temperature swing adsorption unit as a purge gas.

10. The carbon dioxide capture and conversion system of claim 1, further comprising a condenser fluidly coupled to the zeolite membrane reactor and configured to recover water from a stream comprising water and nitrogen.

11. A method of capturing and converting carbon dioxide, the method including:
providing an input stream comprising carbon dioxide to a temperature swing adsorption unit;
separating the input stream comprising carbon dioxide into a carbon dioxide-rich stream and a carbon dioxide-lean stream;
providing the carbon dioxide-rich stream and hydrogen gas to a zeolite membrane reactor; and
hydrogenating the carbon dioxide in the carbon dioxide-rich stream to yield a retentate stream comprising methanol and a permeate stream comprising water and nitrogen gas.

12. The method of claim 11, wherein the input stream comprises flue gas.

13. The method of claim 11, wherein the carbon dioxide-rich stream further comprises carbon monoxide and additional hydrogen gas.

14. The method of claim 11, further comprising separating the retentate stream into a product stream comprising methanol and a recycle stream comprising carbon dioxide, carbon monoxide, and hydrogen gas.

15. The method of claim 14, further comprising providing the recycle stream to the temperature swing adsorption unit as a purge gas.

16. The method of claim 11, further comprising separating the permeate stream into a stream comprising water and a stream comprising nitrogen gas.

17. The method of claim 11, further comprising providing the carbon dioxide-lean stream to the zeolite membrane reactor as a sweep gas.

18. The method of claim 11, further comprising producing the hydrogen by electrolysis.

19. The method of claim 18, wherein producing the hydrogen comprises powering the electrolysis with a renewable energy source.

20. A zeolite membrane reactor comprising:
a housing;
a plurality of hollow fiber zeolite membrane tubules arranged in the housing, wherein each hollow fiber zeolite membrane tubule of the plurality of hollow fiber zeolite membrane tubules defines an annulus; and
a carbon dioxide hydrogenation catalyst contained in the annulus.
